(19) United States
(12) Patent Application Publication
(10) Pub. No.: US 2002/0042929 A1
(43) Pub. Date: Apr. 11, 2002

(54) METHOD OF CREATING TRANSFORMED MAIZE

(76) Inventors: GURDIP S. BRAR, MIDDLETON, WI (US); BARRY A. COHEN, MADISON, WI (US)

Correspondence Address: QUARLES & BRADY LLP, FIRSTAR PLAZA, ONE SOUTH PINCKNEY STREET, O,O, BOX 2113 SUITE 600, MADISON, WI, 53701-2113, US

(21) Appl. No.: 08/888,459

(22) Filed: Jul. 7, 1997

Related U.S. Application Data
(63) Continuation of application No. 08/444,475, filed on May 19, 1995, now abandoned.
Application No. 08/444,475 is a continuation of application No. 07/708,596, filed on May 31, 1991, now abandoned.

(51) Int. Cl. A01H 1/00

(52) U.S. Cl. 800/278

(57) ABSTRACT

A method of creating a transformed maize plant is disclosed. One embodiment of this method includes the steps of isolating a maize embryo, culturing that embryo to produce callus cells, and bombarding the callus cells with particles coated with copies of a nucleic acid construct. In another embodiment of the invention, the cultured embryo produces a mixture of embryogenic and organogenic callus. The nucleic acid-coated particles bombard this mixture, and a transformed plant is produced.

### FIELD OF THE INVENTION

The present invention relates to the field of plant genetic engineering. In particular, the present invention relates to the genetic transformation of maize by nucleic acid-coated accelerated particles.

### BACKGROUND OF THE INVENTION

One general object of modern biotechnology is to genetically engineer crop plants by introducing new genetic traits into the genome of elite plant lines. Plants with new traits, such as insect resistance or herbicide resistance, and artificial manipulations of the agronomic qualities of the crop product, are possible once recombinant genes are introduced into plant lines.

The first widely used plant genetic engineering technique was based on the natural ability of the soil-dwelling microorganism Agrobacterium tumefaciens to introduce a portion of its DNA into a plant cell as a part of the normal pathogenic process. If a foreign gene is inserted into the bacteria in certain ways, the Agrobacterium can be used to transfer the foreign gene into a plant. Agrobacterium transformation techniques have been developed for a number of plants, mostly dicotyledonous, but the usefulness of the technique has varied from plant species to species. Agrobacterium-based transformation systems are limited because they require cell or tissue culture and plant regeneration techniques. Plant lines vary in their amenability to tissue culture and regeneration methods. Regeneration of maize protoplasts has been accomplished recently, but the efficiency of the process is low. Rhodes et al., Bio/Technology 6: 56-60 (1988). A maize genetic engineering system which obviates tissue culture and regeneration would represent a significant advantage.

One new developing technique for creating transformed plants includes bombarding a cell with accelerated particles which carry genetic information. The first indication of the utility of this technique was a demonstration that DNA constructs could be coated onto tungsten particles and accelerated into onion skin, where the genes were transiently expressed, as is described in the specification of U.S. Pat. No. 4,945,050. A problem in the development of an accelerated particle transformation process to create transgenic plants is the difficulty of obtaining a germline plant transformation. By the term “germline transformation” it is meant that the germ cells of the plant are transformed in such a way that the progeny of the plant inherit the inserted foreign genetic construct. In some species, plant genetic transformation has been achieved by the accelerated particle method. European Patent Application No. 301,749 discloses the germline transformation of soybean plants and plant lines. The method disclosed in that published patent application is based on accelerating DNA-coated particles into the excised embryonic axes of soybean seeds. If the bombarded soybean embryonic axes are treated with high cytokinin media, shoots are induced from the treated embryonic axes. When the shoots are cultivated into whole soybean plants, a significant percentage of the plants will have transformed germlines.

Accelerated particle transformation has also been successful in treatment of maize suspension cultures. Fromm et al., Bio/Technology 8: 833-839 (1990), discloses the creation of transformed maize plants from suspension cultures and from calli created from suspension cultures. Klein et al., Pro. Natl. Acad. Sci. USA. 85: 4305-4309 (1988), discloses accelerated particle transformation of maize suspension culture cells. Spencer et al., Theor. Appl. Genet. 79: 625-631 (1990), discloses stable transformed maize callus recovered from suspension culture cells bombarded with DNA-coated accelerated particles. Gordon-Kamm et al., The Plant Cell 2: 603-618 (1990), discloses microprojectile bombardment of embryogenic maize suspension cultures. None of these disclosures deal with transformation of tissues not in cell culture. Most elite lines of maize cannot presently be regenerated from suspension cell cultures. Hence, prior techniques may be limited to certain less desirable genotypes of maize plants.

Maize embryos have been bombarded and transient expression achieved by DNA-coated particles. Klein et al., Bio/Technology 6: 559-563 (1988), discloses particle bombardment of surface cells of an excised maize embryo. This reference does not disclose regeneration from the bombarded embryo, nor inheritance in progeny plants. The cells were bombarded with DNA encoding the beta-glucuronidase (GUS) gene. Because it turns a substrate, 5-bromo-4-chloro-3-indolyl glucuronidide, blue in an in situ tissue assay, beta-glucuronidase can be histochemically detected. After bombardment and treatment with the GUS substrate, blue spots appeared on the embryo, indicating transiently transformed cells.

The art of plant genetic engineering lacks a method of bombarding a maize embryo with nucleic acid-coated particles and producing progeny plants with transformed germline.

### SUMMARY OF THE INVENTION

The present invention is a method of creating transformed maize plants. The method begins with the preparation of copies of a nucleic acid construct. These copies are coated onto biologically inert carrier particles. The nucleic acid-coated carrier particles are physically accelerated toward isolated immature maize embryos. The bombarded embryos are cultivated to produce shoots. These shoots are cultivated into whole sexually mature plants. The presence of the nucleic acid construct is verified either in the shoots or the sexually immature plants.

In a particularly advantageous embodiment of the present invention, the cultured embryos produce a mixture of organogenic and embryogenic callus. The nucleic acid-coated particles bombard this mixture of embryogenic and organogenic callus and transformed plants are produced.

One object of the present invention is to produce a transformed maize plant.

Another object of the present invention is to produce a maize plant with a transformed germline.

Other objects, advantages, and features of the present invention will become apparent from the following specification when taken in conjunction with the accompanying drawings.

### DESCRIPTION OF THE FIGURES

FIG. 1 is an exploded schematic view of a particle acceleration device useful in the present invention.

FIG. 2 is a top plan view of the device of FIG. 1.

FIG. 3 is an illustration of the plasmid TV5-HS-GUS used in the Example.

FIG. 4 is an enlarged drawing of the corn immature embryo stage 1.

### DESCRIPTION OF THE PREFERRED EMBODIMENT

The present invention is a method of obtaining germline transformed maize plants. In this method, immature maize embryos are first isolated and cultured. It is envisioned that most maize varieties will be amenable to the present invention because the culture characteristics of embryos and scutella culture from different maize inbred varieties or lines are similar. Scutella culture, as described below, may be applied to most maize inbred lines. Second, the cultured embryos are bombarded with nucleic acid-coated particles, and transformed plants are regenerated from the bombarded cells. The regeneration protocol is based on somatic embryogenesis. From plants recovered from the somatic embryogenesis process, it has been surprisingly discovered that clonal germ line transformed maize plants can be recovered.

A. Preparation of Maize Embryos

Maize kernels are sterilized and isolated from conditioned ears. Preferably, the ears from which these kernels are taken have been conditioned at 4° C. from one to eight days with four to six days being preferred. The maize embryo, which is composed of the scutellum and the shoot/root axis, is then excised from the kernel. The scutellum is the cotyledon of the maize embryo. Esau, Anatomy of Seed Plants, John Wiley & Sons, New York, p. 477. Preferably, immature embryos are excised. By “immature” we mean that the embryos are approximately between 0.50 mm and 2.50 mm in length are in the developmental stages early coleoptilar, stage one or stage two, as defined by Abe and Stein Am. Jour. Bot. 41, pp. 285-293 (1954). The embryos just entering stage one are preferred. Mature embryos may be used in the present invention, but it is more difficult to culture these embryos. Very immature embryos may be used, but these smaller embryos are harder to manipulate.

The maize embryos are placed on a medium amenable to immature embryo scutella culturing. The embryonic axis should touch the media, and the scutellum should be exposed. Preferably, the medium should contain the salts and carbon source (sucrose or other sugars) needed for growth, an auxin or auxin-like substance, and agar or other gelling agent. In the practice to date, the culturing medium contained chloramben, an auxin. Other auxins, such as 2,4-D may also be used. The embryos are incubated in the dark until callus cells form. Usually, this takes about four days. By “callus cells,” it is intended to describe undifferentiated cells induced to form from the scutella tissue.

Preferably, the callus that develops on the scutellum is a mixture of organogenic and embryogenic callus. Almost every maize line is susceptible to this type of callus culture. For example, McCain and Hodges, Bot. Gaz. 147[4]: 453-460 (1986), disclose a method of obtaining somatic embryos that have developed from scutellum of immature zygotic embryos of maize variety A188.

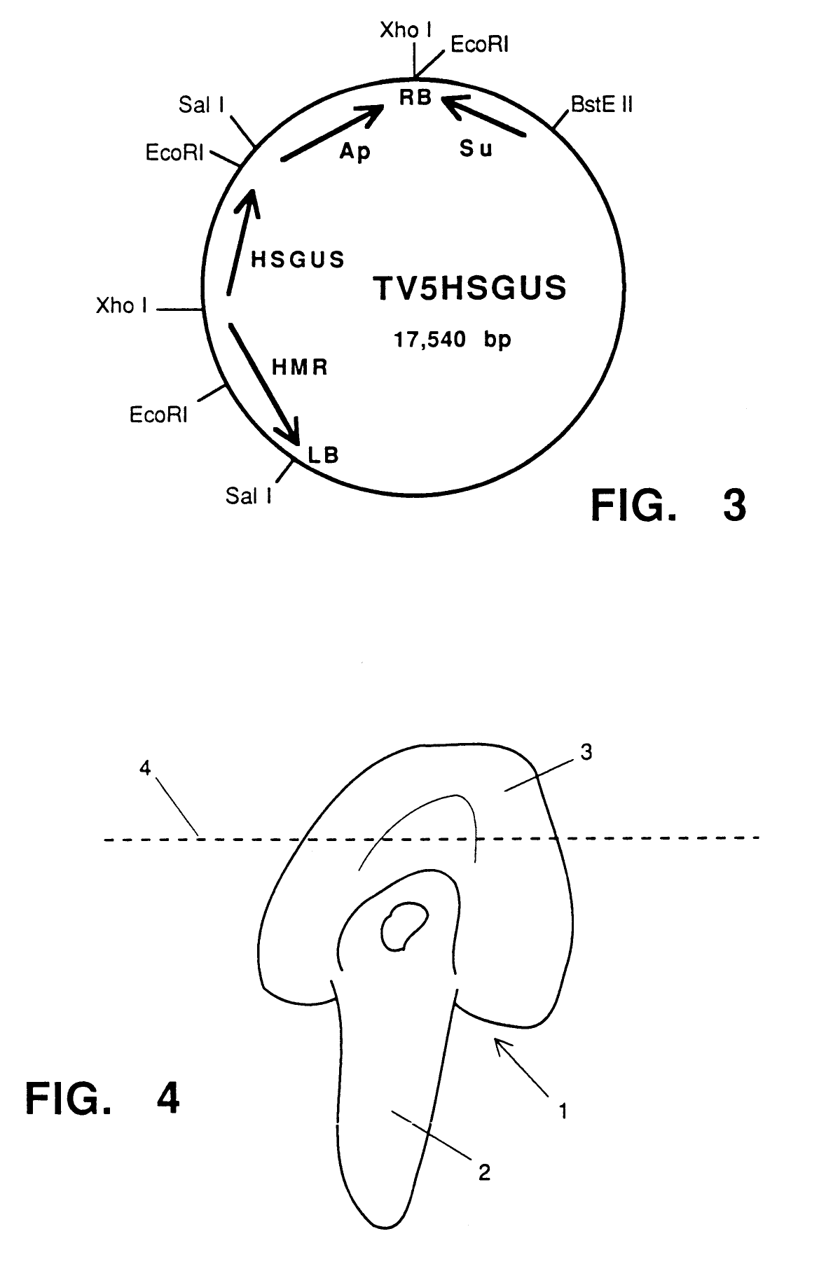

In transformation efforts using Agrobacterium-based procedures with other crops, it has been found that wounded tissue often gave a better transformation result. This phenomenon has not been demonstrated to be useful in accelerated particle transformation methods. Nevertheless, in the protocol used here a part of the embryo that is not producing callus is preferably excised to optimize transformation results. The nonproducing tissue is visually identified by the lack of visible callus proliferation. Typically, the callus proliferates mainly at the suspensor end of the immature embryo on abaxial side of scutellum. The portion of the embryo that is removed is from the coleoptile end. This is illustrated in FIG. 4, illustrating a stage 1 embryo modelled after Abbe and Stein, Am. Jour. Bot., 41, pp. 287 (1954). The suspensor end 2 is where the callus proliferates on the lower one-half to three-quarters of the scutellum. The top one-half to one-quarter of the coleoptile end 3 of the scutellum is the part that is removed. A dashed line 4 indicates the usual locus of the art. The removed scutellum portion is discarded.

The dissected embryos are placed on fresh medium and positioned so that the callus tissue is available to be bombarded by the accelerated particles. Preferably, this medium now contains an osmoticum such as 0.4 M mannitol, so that the embryogenic cells will be partly plasmolized. This osmotic condition helps to preserve cell integrity through bombardment. Optimally, the embryos should remain on this medium one to three hours at room temperature in the dark before they are bombarded

B. Preparation of Nucleic Acid-Coated Particles

Multiple copies of the nucleic acid construct, either RNA or DNA, are prepared by known molecular biology techniques. By “nucleic acid constructs” we mean any RNA or DNA molecule capable of functioning within a maize cell. A suitable nucleic acid construct might be an isolated gene with accompanying regulatory signals or might be a population of RNA or DNA molecules. The nucleic acid might originate in maize or in any other species.

To be useful in a particle-mediated transformation process, the nucleic acid construction must be capable of performing some useful function in the cells of target plant tissues. The transforming genetic construction will normally be a chimeric construction in the sense that its genetic material originates from more than one kind of organism. The genetic construction could be one that is capable of expressing a gene product in the target tissues. Such gene products will typically be a foreign protein but could be other gene products as well, as such as an antisense RNA construct intended to inhibit an endogenous plant system.

Foreign genetic constructions are often embodied in expression cassette vectors for plant cells, many of which are known in the art. Typically such a plant expression vector system includes the coding sequence for the desired foreign gene and appropriate regulatory sequences. The appropriate regulatory sequences might include a promoter sequence capable of initiating transcription and a translational terminator. Some promoters and transcription terminators found to be effective in other plants are effective in maize as well. A translation or transcriptional enhancer may be incorporated between the promoter and the coding region of the genetic sequence.

The transforming nucleic acid construct can include a marker gene which can provide selection or screening capability in the treated plant tissues. Selectable markers are generally preferred for plant transformation events, but are not available for all plant species. A selectable marker encodes for a trait in the transformed plant cells which can be selected for by the exposure of the plant tissues to a selection agent. Suitable selectable markers can be antibiotic or herbicide resistant genes which, when inserted in some cells of a plant in culture, would imbue those particular cells with the ability to withstand exposure to the antibiotic or the herbicide.

It has been found here that at least one antibiotic selectable marker system can be made to work in maize. A gene coding for resistance to the antibiotic hygromycin from E. coli has previously been found to be useful as a selectable marker in tobacco transformation. Waldron et al., Plant Mol. Biol., 5:103-108 (1985). Such selectable markers do not reliably transfer from species to species, particularly when used on callus or differentiated tissue as opposed to protoplasts or suspension culture. Hygromycin resistance has not previously been demonstrated to be effective in maize as a selectable marker.

Another type of marker gene is one that can be screened by histochemical or biochemical assay, even though the gene cannot be selected for. A suitable marker gene found useful in such plant transformation experience is the GUS gene, discussed above. Jefferson et al. EMBO J., 6:3901-3907 (1987) discloses the general protocol for a GUS assay. Thus, the use of a GUS gene provides a convenient calorimetric assay for the expression of introduced DNA in plant tissues by histochemical analysis of the plant tissues. In a typical transformation process, the gene of interest sought to be expressed in the plant can be coupled in tandem with the GUS gene. Then the tandem construct can be transformed into plant tissues and the resulting plant tissues can be analyzed for expression of the GUS enzyme. Tissues and plants expressing the GUS enzyme will also be found to be expressing the gene of interest.

The transformation process requires carrier particles of a durable, dense, biologically-inert material. Gold is a suitable material for use as the carrier particle. The carrier particles are of extremely small size, typically in a range of one to three microns, so that they are small in relation to the size of the maize target cells. Preferably, microcrystalline gold particles are used as carrier particles. A suitable source of microcrystalline gold particles is “Gold A15701” from Engelhart Corporation of East Brunswick, N.J. This product consists of gold particles of high surface area and amorphous shape and size. We found that microcrystalline carrier particles of irregular size achieve a higher transformation efficiency than that achieved by spherical gold particles.

The genetic material to be inserted into the cells is coated onto the carrier particles. This can be readily done by drying solutions of DNA or RNA onto the carrier particles themselves. Suitable stabilizers can be added to the mixture to help with the longevity of the genetic material on the carrier particles, such as the preparation based on spermidine described in the Example below.

C. Bombarding Cultured Embryos

The apparatus utilized in the present invention must be capable of delivering the nucleic acid-coated particles into plant cells in such a fashion that a suitable number of cells can be transformed. At some frequency, the carrier particles lodge within the maize cells and, through a process we do not understand, the genetic materials leaves the carrier particles and integrates into the DNA of the host maize cells. Many types of mechanical systems can accelerate the carrier particles into plant cells. Possible mechanisms include ballistic explosive acceleration of particles, centrifugal acceleration of particles, electrostatic acceleration of particles, or other analogous systems capable of providing momentum and velocity to small particles.

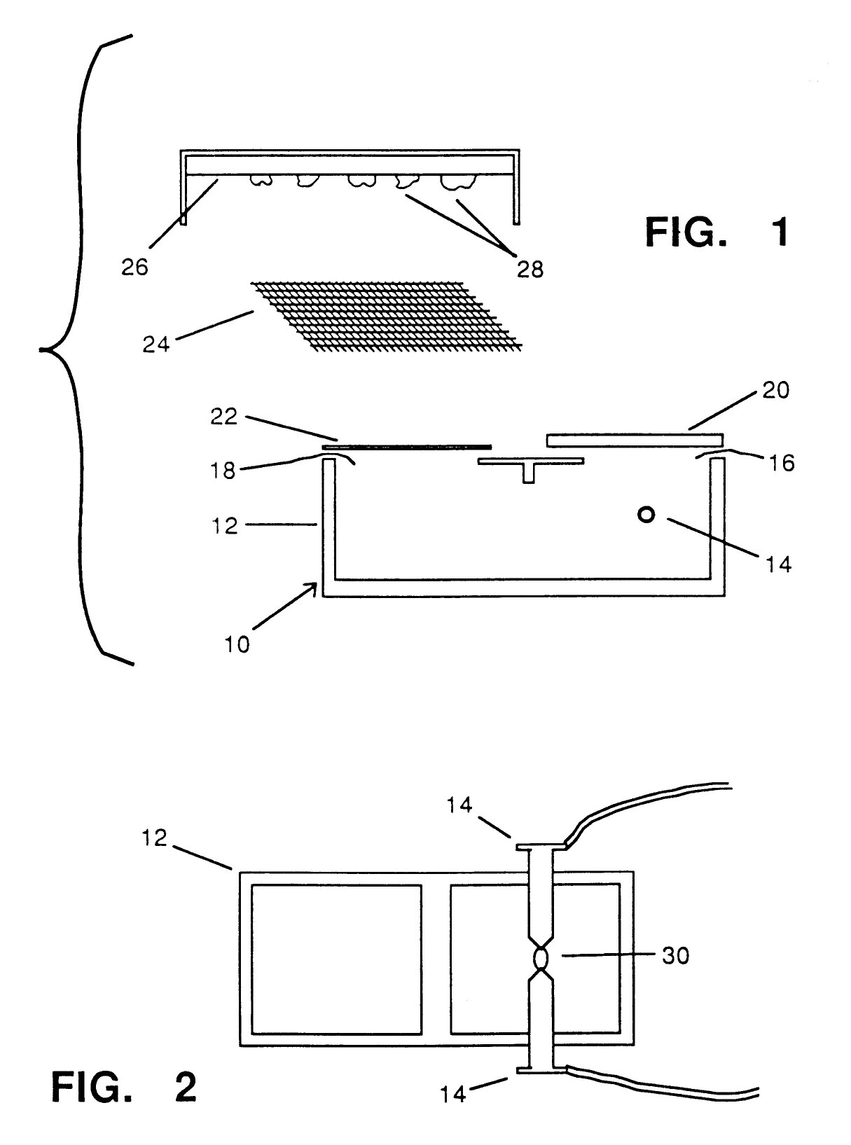

The mechanism we used in the example is based on the acceleration of particle through an adjustable electric voltage spark discharge device which is capable of accelerating a planar carrier sheet at a target surface. This apparatus will be described further below with reference to FIGS. 1 and 2.

The particle acceleration apparatus is generally indicated at 10 of FIG. 1. The apparatus consists of the spark discharge chamber 12 into which are inserted two electrodes 14 spaced apart by a distance of approximately one to two millimeters. The spark discharge chamber 12 is a horizontally extended rectangle having two openings, 16 and 18, extending out its upward end. The opening 16 is covered by an access plate 20. The opening 18, located on the side of the rectangle of the spark discharge chamber opposite from the electrode 14, is ultimately intended to be covered by a carrier sheet 22.

The electrodes 14 are connected to a suitable adjustable source of electric discharge voltage (not shown). A suitable source of electric discharge voltage includes a capacitor in the size range of one to two microfarad. The voltage of the charge introduced into the capacitor should be adjustable. An adjustable voltage can be introduced readily into such a capacitor through the use of an autotransformer which can be adjustable between a range of one and fifty thousand volts. Preferably, a high voltage electric switch is provided so that the capacitor can be discharged safely through the electrodes 14 without harm to the operator.

A carrier sheet 22 is placed upon the opening 18 of the spark discharge chamber 12. The carrier sheet 22 is a planar sheet of relatively stiff material which is capable of carrying small, inert carrier particles thereon toward the target surface. Preferably, the carrier sheet 22 is a small sheet of aluminized, saran-coated mylar. We envision that other relatively stiff, planer materials may be used for the carrier sheet 22. The function of the carrier sheet 22 is to convert an outwardly outstanding force produced by the electrodes to a broadly distributed horizontal force capable of accelerating a large number of carrier particles in parallel with an even force. Other kinds of force other than electric discharge can be used to propel the carrier sheet 22 upward. The force should be adjustable so that the force of travel of the carrier sheet 22 can be adjusted.

Again referring to the apparatus of FIGS. 1 and 2, a retaining screen 24 is approximately 15 millimeters above the opening 18 and the discharge chamber 12. A target surface 26 is placed above the retaining screen 24 at a distance of between 5 and 25 millimeters. The target surface 26 is any suitable culture surface onto which the material to be transformed can readily be placed. An overturned petri dish can conveniently be used for the transformation of plant tissues. Using a semisolid or solid agar-based medium in the bottom of a petri dish, it is possible to place tissues on the agar where they will be retained. The petri dish itself can serve as the target surface while retaining the plant tissues on the agar.

The DNA-coated particles are layered onto the top of the carrier sheet 22. The layering is done so as to distribute a relatively even pattern of carrier particles across the entire top surface of the carrier sheet 22. Preferably, the coated carrier particles are applied to the carrier sheet at a loading rate of 0.025 to 0.050 milligrams of coated carrier particles per square centimeter of carrier sheet. The carrier sheet 22 is placed upon opening 18. An oil or water droplet is used to adhere the carrier sheet 22 in place. The target surface 26, with the living plant material thereon, is placed in position above the retaining screen 24. A small droplet of water, preferably 10 microliters, is placed in the chamber bridging between the ends of the two electrodes 14. The access cover 20 is placed in position on top of the spark discharge chamber 12.

At this point, the entire apparatus is enclosed in a vacuum chamber and a vacuum is drawn down into the range of approximately 500 millimeters of mercury. As the vacuum is drawn, a supply of helium is bled into the vacuum chamber. Thus, the vacuum chamber contains a relative vacuum compared to the atmosphere and the atmosphere within the vacuum contains helium. The lower density of helium, combined with the reduced pressure, lowers the drag on both the carrier sheet 22 and the carrier particles.

The accelerated particle transformation process is initiated at this point. The voltage from the capacitors is electrically discharged to the electrodes 14. The voltages used in the present process have been in the range of 10-25 kV. The range of 16-18kV is preferred. The voltage is discharged through the use of appropriate electric switching described above. The force of the electric discharge initiates a spark which leaps the gap between the electrodes 14 and vaporizes the small droplet of water which was placed between the electrodes. The vaporization force creates a violent atmospheric shock wave within spark discharge chamber 12. The shock wave radiates outward from the electrodes in all directions. Because of the immovable sides of the chamber, the impact of the radiating shock wave upon the interior of the discharge chamber 12 is focused towards the carrier sheet 22, which is then propelled upward with great velocity. The upwardly traveling carrier sheet 22 accelerates upward at great force until it contacts the retaining screen 24. The displacement of the remaining atmosphere in the chamber with helium assists in the travel of the carrier sheet 22, since helium provides less drag on the flight of the carrier sheet as well as on the carrier particles themselves. At the retaining screen 24, the carrier sheet 22 impacts the retaining screen 24 and is retained. The nucleic acid-coated particles, in contrast, fly off of the carrier sheet and travel freely toward the target tissues. The small carrier particles then hit the living tissue on the target surface and proceed into the tissue cells.

D. Regeneration of Maize Plants

Plants must be created from the bombarded callus tissue. At either the cellular or plant level, the plants must be screened or selected to segregate the transformed plants from the nontransformed plants. In most particle-mediated plant transformation procedures done without selection, the nontransformed plants will be the large majority of the recovered plants. If a selection agent like hygromycin is used, the number of plants recovered is smaller but the relative proportions of transformant plants recovered is higher. The selection regimen may not kill all of the non-transformed embryogenic cells, but it has been found to be useful in enriching the pool of recovered plants so that germ line transformant plants are recovered with greater frequency.

After bombardment, the embryo tissue is fragile. It is considered advantageous to let the bombarded tissues remain on the culture medium for approximately four hours. If a gene encoding a selection marker has been incorporated into the cells, the regenerating plant must be placed on medium containing the selection agent at some point in the growth process.

First, shoots must be induced from the transformed cells. Shoot induction methods are well-known in the art. Green and Phillips, Crop. Sci. 15:417-420 (1975) disclose one practical method for regenerating maize shoots from callus culture. The Example discloses a shoot induction protocol that involves incubation on media with hygromycin, kinetin, and IAA. Green callus tissue appears, and this tissue is transferred to another medium, MS-hygromycin, for further growth. Second, mature plants must be grown from the shoots. In the Example, plantlets are transferred to rooting medium for 15 days and then transferred to acclimated in the mist bench, and then transferred to normal greenhouse conditions for growing corn.

### EXAMPLE 1

510 nine day old immature embryos were excised from kernels of maize inbred line A634 and plated on N6-CP medium. The kernels came from ears that had been conditioned at 4° C. for six days before use. N6-CP medium contains, per liter, 4 g of N6 salts (sigma chemical), 20 g of ultra pure sucrose, 100 mg of lactalbumim hydrolysate, 2.3 g of L-proline, 10 ml of chloramben (0.228 mg/ml) and 8 g of agar at pH 5.8. This medium, and all other media mentioned below, was autoclaved at 121° C. for 25 minutes prior to use. Some additives, such as vitamins or hygromycin were added after sterilization and were separately filter sterilized. The N6 vitamins added to N6-CP medium, per liter, were: 2 mg of glycine, 1 mg of thiamine Hcl, 0.5 mg of pyridoxine Hcl and 0.5 mg of nicotinic acid. The excised embryos were approximately 1.5 mm long (±0.25 mm). The embryos were incubated at 28° C. in the dark for four days.

Incubation in the dark initiated a combination of embryogenic and organogenic callus from the embryo scutella. In preparation for bombardment, the part of the embryo that was not producing callus was excised and discarded as discussed above. The embryos were plated on N6-CP medium containing 0.4 molar mannitol for 1 to 2.5 hours at room temperature in the dark. The embryos were positioned such that the DNA-coated beads could hit the callus cells.

The nucleic acid construct we used was the plasmid TV5-HS-GUS, diagramed in FIG. 3. This construct contains genes encoding both hygromycin resistance and GUS. The hygromycin gene (HMR) includes a gene coding for hygromycin resistance (encoding hygromycin phosphotransferase) behind a cauliflower mosaic virus 35S promoter (CaMV35S) and in front of a nopaline synthase polyadenylation sequence from Agrobacterium tumefaciens (poly A). The GUS gene construct (HSGUS) includes the beta-glucuronidase enzyme coding sequence behind a heat-shock promoter and in front of a nopaline synthase poly A. The plasmid also includes the left and right border sequences (LB and RB) from an Agrobacterium Ti plasmid, but these sequence are not believed important and are merely an artifact of the method of construction of the plasmid.

Plasmid DNA was isolated by standard molecular biological methods. The DNA was coated onto gold beads using a spermidine-PEG-CaCl₂ protocol as follows: 10 mg of amorphous microcrystalline gold was measured into the bottom of a 1.5 ml Eppendorf microfuge tube. Care was taken to ensure that the gold did not spill on the sides of the tube, since that would make it difficult to resuspend the gold in the small volumes used in the preparation process. 100 μl of H₂O was added, and the tube was vortexed gently. 10.0 micrograms of plasmid DNA was added to the microfuge tube, and the tube was vortexed gently for 5-10 seconds. 100 μl of 0.1 M spermidine solution (free base) was added to this microfuge tube, and the microfuge tube was vortexed. 100 μl of 25% PEG solution (MW 3000-4000) is added, and the tube was vortexed well. While the DNA/carrier particle/PEG mixture was vortexed, 100 μl of 2.5 M CaCl₂ was added drop by drop to the tube. The vortex was stopped, and precipitation was permitted for 10 minutes at room temperature. The preparation could be stored at this point for some time. Shortly before use, the mixture of DNA and carrier particles was given a brief spin in a microfuge. The cleared supernatant was removed completely, and the particles were washed twice in 500 μl of ethanol. The precipitate, consisting of the DNA and carrier particles, was resuspended in 10 mls of 100% ethanol. The resuspended DNA and carrier particle mixture was sonicated in a water bath sonicator for two to three one second exposures. The resulting suspension was then coated onto an 18×18 mm carrier sheet at a rate of 163 μl per carrier sheet, or a calculated rate of 0.05 milligrams per square centimeter of the carrier sheet. In summary, the gold particles were coated with DNA at a level of approximately 1 μg DNA/1 mg particles, and the particles were resuspended at a concentration of 1 mg particles/1 ml ethanol.

The carrier sheets were placed upon the apparatus of FIGS. 1 and 2 for the blasting process. The cultured embryos were plated in a petri dish on N6-CP media with 0.4 M mannitol. Typically, 30 cultured embryos were placed on the dish and bombarded. The target plates were 60×15 mm and contained 10 ml of media. The embryos were incubated on the media in the dark at room temperature for 1-2.5 hours before bombardment.

The parameters used to bombard the maize embryos were as follows: The voltage discharged through the apparatus was 18 kV. 50% of the plates were bombarded twice with a 2.5 hour rest between treatments. Between treatments, the plates were kept at room temperature in the dark. The chamber was at 508 millimeters of mercury, with helium introduced at a rate of 1.5 liters per minute at atmospheric levels and approximately 5.0 liters per minute under the vacuum.

The bombarded embryos remained on the media on the target surface for four hours at room temperature in the dark. The plates were inverted such that the embryos were hanging from the agar surface. The bombarded embryos were then transferred to N6-CP media containing 0.1 M mannitol and incubated at 28° C. in the dark for seven days. The germinating embryo shoot axes were sliced off, and the tissues were transferred to N6-CP hygromycin medium (N6-CP medium with 50 mg of hygromycin added per liter) at 28° C. in the dark for two weeks. Once during the time period, the culture was re-plated.

The tissues were transferred to W+ hygromycin medium at 25±1.5° C. with diffused light to full light, with 16 hour light and 8 hour dark photoperiod with cool white fluorescent lighting. W+ hygromycin medium is, per liter, 4.31 grams of MS salts (Gibco), 30 grams of sucrose, 0.1 gram of myo-inositol, 3 mg of glycine, 0.5 mg of nicotinic acid, 0.1 mg of pyridoxin HCl and 0.1 of thiamin HCl, 0.2 grams of Adenine sulfate, 0.34 gram of sodium phosphate monobasic, 0.05 gram of benomyl, 3 ml of kinetin (1 mg/ml), 8 g of agar, and 2 ml of IAA (1 mg/ml) at pH 5.6, to which was added 50 mg/liter hygromycin. In this culture step, several resistant calli started to turn green.

The green callus tissue was transferred to MS-hygromycin medium. MS-hygromycin medium is, per liter, 4.31 g of MS salts, 3 mg of glycine, 0.5 mg of nicotinic acid, 0.1 mg of pyridoxin HCl, 0.1 mg of thiamin HCl, 20 g of sucrose, 0.1 g of myo-inositol, 8 g of agar at pH 5.8, and 25 or 50 milligrams per liter hygromycin. One plant, 369-17, survived selection and was assayed for GUS expression and the presence of the hygromycin resistance gene. Two independent samples were taken from the plant. Hygromycin resistance gene primers specific for the 5′-end of the gene, the 3′-end of the gene, and a combination of both parts were employed in the polymerase chain reaction (PCR) analysis. PCR assays were performed twice for each of the two samples and verified the presence of the hygromycin resistance gene. Histochemical assays confirmed the presence of the GUS gene.

The plant was transferred to rooting medium at 25±1.5° C. in light conditions (16 hour photoperiod). Rooting medium is, per liter, 4.31 g MS salts, 3 mg of glycine, 0.5 mg of nicotinic acid, 0.1 mg of pyridoxin HCl, 0.1 mg thiamin HCl, 20 g Sucrose, 0.1 g Myo-inositol and 8 g of agar. After fifteen days rooting medium, the plant was placed in Metromix (W. R. Grace) at 25±1.5° C. in light conditions (16 hour photoperiod). The plant was acclimated in the mist bench for 3 days and then transferred to the normal greenhouse conditions for growing corn. All the leaves of the grown plant tested positive in a PCR assay for the hygromycin resistance gene. In a histochemical assay, all the leaves of the plant tested positive for expression of GUS. Also in a histochemical assay, the immature pollen of the mature plant was positive for the GUS gene at approximately a 1:1 ratio. This ratio is a good indication of a single gene insertion. However, mature corn pollen did not test positive. We speculate that since corn pollen is trinucleate and the nuclear divisions are complete, GUS translation mechanism may be impeded at pollen maturity.

Next generation progeny from this plant have been recovered. Of 234 progeny (R1) plants resulting from a cross between this plant and non-transgenic stock, 112 leaves which tested positive for GUS in their leaves. Of the 10 R1 progeny resulting from self-pollination which were tested, 8 were GUS positive. Both results are a good match with expected Mendelian segregation (1:1 and 3:1). This demonstrates the expected genetic inheritability of the inserted nucleic acid construct and confirms germ line transformation.

### EXAMPLE 2

324 ten-day-old immature embryos were excised from kernels of maize inbred line A634 and plated on N6CP medium for four days at 28° C. dark. The kernels came from ears that had been conditioned at 4° C. for four days before use. Excised embryos were 1.5 mm±0.25 mm. Before blasting, the part of embryos not producing callus was cut off, as in Example 1, and plated on N6CP 0.4M mannitol medium for about one and one-and-a-half hours. After blast, the embryos stayed on N6CP 0.4M mannitol medium for about 5 hours instead of 4 hours in Example 1, at room temperature, in the dark. The tissues were transferred to N6CP hygromycin at 50 mg/l medium after only six days on N6CP 0.1M mannitol. After eight days on N6CP hygromycin 50 mg/l, the embryos with callus were transferred to N6CP hygromycin at 25 mg/l. After seven days the callus producing embryos were transferred to W+ medium without any hygromycin selection followed by transfer to MS with hygromycin at 25 mg/l for 5 days, to W+ hygromycin (50 mg/) for thirteen days to MS with hygromycin (25 mg/1) for eight days and then to the rooting medium. Only one plant, 402-6, survived this selection. A part of each of its four leaves was GUS assayed to determine the integration and expression of GUS gene. A strong GUS expression was observed in all leaf segments. Plant has been rooted and is showing good growth in the greenhouse. DNA construct used, blasting parameters and all other details not referred to above were identical to Example 1.

### EXAMPLE 3

175 ten-day-old embryos were excised from surface sterilized kernels of maize inbred line A634 and plated on N6CP medium at 28° C., dark for four days. The kernels came from ears that had been conditioned at 4° C. for three days before use. The immature embryo scutella size was 2 mm±0.25 mm.

DNA constructs used, preparation of embryos for blasting, pre-blast conditioning on N6CP 0.4M mannitol medium, blasting parameters, post-blast treatment on N6CP 0.4M mannitol and post-blast transfer to N6CP 0.1M mannitol medium were similar to Example 1. After one week on N6CP 0.1M mannitol medium the callused embryos were transferred to N6CP with hygromycin (50 mg/l) for ten days followed by transfer to N6CP with 25 mg/l of hygromycin for seven days, all at 28° C., dark. The embryos with organogenic/embryogenic callus were transferred to W+ with hygromycin (50 mg/l) for eight days (diffused light to 16 hour light) and then to MS with hygromycin (25 mg/l) for eight days.

Single plant, 405-4, survived this selection. A part of each of its four leaves, assayed for GUS, showed very strong GUS expression uniformly in all areas suggesting the stable integration and expression of GUS gene.

This plant is now on rooting medium.

We claim:

1. A method of creating a transformed maize plant comprising the steps of:
(a) preparing copies of a nucleic acid construct;
(b) coating the nucleic acid construct copies onto biologically inert carrier particles;
(c) isolating a maize embryo;
(d) culturing the maize embryo, such that at least a portion of the embryo produces callus cells;
(e) placing the cultured embryo on a target surface;
(f) accelerating into the cultured embryo the nucleic acid-coated carrier particles in such a fashion that some particles lodge in the interior of at least some of the embryo cells;
(g) cultivating the bombarded embryo such that shoots arise from the bombarded embryo;
(h) cultivating the shoots formed in step (g) into whole sexually mature plants; and
(i) verifying the presence of the nucleic acid construct in the shoots formed by step (g) or the plants formed by step (h).

2. The method of claim 1 wherein step (j) is added:
(j) verifying the inheritability of the nucleic acid construct.

3. The method of claim 1 wherein at least a segment of the cultured embryo produced in step (d) is excised before the bombardment of step (f).

4. The method of claim 3 wherein the excised segment is from a portion of the cultured embryo that has not produce callus cells.

5. The method of claim 1 wherein the cultured embryo of step (d) produces a mixture of organogenic and embryogenic callus.

6. The method of claim 1 wherein the length of the excised embryo is approximately 0.5 to 2.5 mm.

7. A method of creating a transformed maize plant comprising the steps of:
(a) preparing copies of a nucleic acid construct;
(b) coating the nucleic acid construct copies onto biologically inert carrier particles;
(c) isolating a maize embryo;
(d) placing the embryo on a target surface;
(e) accelerating at the embryo the nucleic acid-coated carrier particles in such a fashion that some particles lodge in the interior of at least some of the embryo cells;
(f) cultivating bombarded embryo such that shoots arise from at least some of the bombarded embryos;
(g) cultivating the shoots formed in step (f) into whole sexually mature plants; and
(h) verifying the presence of the nucleic acid construct in the shoots formed by step (f) or the plants formed by step (g).

8. The method of claim 7 wherein step (i) is added:
(i) verifying the inheritability of the nucleic acid construct.

9. The method of claim 7 wherein at least a portion of the embryo of step (c) is excised before the bombardment of step (e).

10. The method of claim 9 wherein the length of the excised embryo is approximately 1.5 mm±0.25 mm.

11. A method of creating a transformed plant comprising the steps of:
(a) preparing copies of a nucleic acid construct including a foreign gene of interest and a selection agent gene;
(b) coating copies of the nucleic acid construct onto biologically inert carrier particles;
(c) conditioning a maize ear below room temperature for one to six days;
d) isolating from the maize ear an immature embryo of between 0.5 and 2.5 mm in length;
(e) incubating the embryo in the dark on a medium incorporating an auxin therein until callus proliferation is induced;
(f) excising the portion of the embryo from which callus does not proliferate;
(g) culturing the remaining embryo tissue in an osmoticum for at least one hour;
(h) accelerating into the remaining embryo tissue the nucleic acid particles in such a way that at least some of the particles lodge in the interior of at least some of the cells in the embryo tissue;
(i) culturing the tissues from step (h) in an osmoticum without selection for a time period sufficient and under conditions sufficient for somatic embryos to begin to arise on the tissues;
(j) removing the somatic embryos from the tissues from step (i) and culturing the embryos in the presence of a selection agent which selects for the presence of the election agent gene in the cells;
(k) regenerating the embryos into plants for those embryos which survive the selection; and
(l) verifying in the plants thus produced the incorporation of the nucleic acid construct.

12. Maize plants comprising in their cells an inheritable trait conferring on the cells a resistance to the antibiotic hygromycin.
